(19) United States
(12) Patent Application Publication
(10) Pub. No.: US 2005/0021235 A1
(43) Pub. Date: Jan. 27, 2005

(54) Method and system for optically performing an assay to determine a medical condition

(76) Inventors: Raphael Bar-Or, Denver, CO (US); David Bar-Or, Englewood, CO (US); C. Gerald Curtis, Penylan Cardiff (GB)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/477,384

(22) PCT Filed: May 30, 2002
(86) PCT No.: PCT/US02/16860

(30) Foreign Application Priority Data
May 30, 2001 (US) 60294955

(51) Int. Cl. G01N 33/48

(52) U.S. Cl. 702/19

(57) ABSTRACT

A method and system are disclosed for detecting a medical condition wherein a blood or plasma sample is combined with a metal such as cobalt and optically analyzed for an optical distinction that identifies the medical condition. The invention is useful for diagnosing medical conditions such as ischemia. Moreover, the diagnoses of patient samples according to the invention may be enhanced by developing a mathematical model based on signal processing techniques such as principal component analysis on the data obtained in patient studies.

### RELATEDNESS OF THE APPLICATION

The subject application claims the benefit of priority from U.S. Ser. No. 60/294,955, filed May 30, 2001, which is incorporated herein in its entirety.

### BACKGROUND

Ischemia is the leading cause of illness and disability in the world. Ischemia is the state of imbalance of oxygen supply and demand in a part of the body often due to a constriction or an obstruction in the blood vessel supplying that part. The two most common forms of ischemia are cardiovascular and cerebrovascular.

Cardiovascular ischemia is generally a direct consequence of coronary artery disease, and is usually caused by rupture of an atherosclerotic plaque in a coronary artery, leading to formation of thrombus (blood clot), which can occlude or obstruct a coronary artery, thereby depriving the downstream heart muscle of oxygen. Prolonged ischemia can lead to cell death or necrosis, and the region of dead tissue is commonly called an infarct. Patients suffering an event of acute cardiac ischemia often present to a hospital emergency room with chest pain and other symptoms and signs (such as changes to the electrocardiogram of ECG) referred to as Acute Coronary Syndromes or ACS. A patient diagnosed with ACS requires immediate treatment to avoid irreversible damage to the heart muscle.

Cerebral ischemia is often due to narrowing of the arteries leading to the brain, and early symptoms may be called Transient Ischemic Attack (TIA), which may include headache, dizziness, sensory changes, and temporary loss of certain motor function. TIAs are a precursor to cerebrovascular accident (CVA) or stroke which is the third leading cause of death in the United States.

The continuum of ischemic disease includes five conditions: (1) elevated blood levels of cholesterol and other lipids; (2) buildup of atherosclerotic plaque and subsequent narrowing of the arteries; (3) reduced blood flow to a body organ (as a result of arterial narrowing or plaque rupture and subsequent thrombus formation); (4) cellular damage to an organ caused by a lack of oxygen; (5) death of organ tissue caused by sustained oxygen deprivation. Stages three through five are collectively referred to as “ischemic disease,” while stages one and two are considered its precursors.

It is important to distinguish between the state of ischemia and the disease which leads to it. For example, a patient with coronary artery disease is not always in the state of cardiac ischemia, but a person in the state of cardiac ischemia almost invariably suffers from coronary artery disease.

Together, cardiovascular and cerebrovascular disease accounted for 954,720 deaths in the U.S. in 1994. Furthermore, more than 20% of the population has some form of cardiovascular disease. It was estimated that in 1998, as many as 1.5 million Americans would have a new or recurrent heart attack, and about 33% of them would die. Additionally, as many as 3 to 4 million Americans suffer from what is referred to as “silent ischemia.” This is a condition where ischemic heart disease is present without the usual and classic symptoms of chest pain or angina.

There is a pressing need for the development and utilization of blood tests able to detect injury to the heart muscle and coronary arteries. Successful treatment of cardiac events depends largely on detecting and reacting to the presence of cardiac ischemia in time to minimize damage. Cardiac enzymes, specifically the creatine kinase isoenzyme (CK-MB), and markers of cardiac necrosis, specifically myoglobin and the Troponin I and Troponin T biochemical markers, are utilized for diagnosing heart muscle injury. However, these enzymes and markers are only capable of detecting the existence of cell death or necrosis, and therefore have limited or no value in patients who have ischemia without necrosis, such as those in an ischemic state prior to myocardial infarction. Additionally, these enzymes and markers do not show a measurable increase until several hours after the onset of necrosis. For instance, the cardiac troponins do not show a measurable increase above normal in a person's blood test until about four to six hours after the beginning of a heart attack and do not reach peak blood level until about 18 hours after such an event. Thus, the primary shortcoming of using markers of cardiac necrosis for diagnosis of ischemic states is that these markers are only detectable after heart tissue has been irreversibly damaged.

A pressing requirement for emergency medicine physicians who treat patients with chest pain and stroke symptoms is for a diagnostic test that would enable them to definitively “rule out” or “rule in” acute coronary syndrome (which may be acute myocardial infarction), stroke, and other emergent forms of ischemia. A need exists for a method for immediate and rapid distinction between ischemic and non-ischemic events, particularly in patients undergoing acute cardiac-type symptoms. While the ACB™ Test (Ischemia Technologies, Inc., Denver, Colo.) is such a test, the medical demand is such that additional diagnostic tests are desirable.

A broad array of diagnostic tests is available for diagnosis of cardiac ischemia, particularly in the emergency room (see, for example, Selker, H P, Zalenski, R J et al An Evaluation of Technologies for Identifying Acute Cardiac Ischemia in the Emergency Department: A Report from a National Heart Attack Alert Working Group Annals Emergency Medicine 1997;29:13-87). The accepted standard of care is the 12 lead electrocardiogram (ECG or EKG) which, nevertheless, has a clinical sensitivity of less than 50%. Other diagnostic tests include echocardiography, and radionuclide myocardial perfusion imaging.

Diagnosis of coronary artery disease is done either by imaging (e.g.: coronary angiography) or by provocative testing, where the intent is to deliberately induce cardiac ischemia and observe the effects. For example, in the ECG exercise stress test, the patient is exercised at an increasing rate to see if symptoms of ischemia are evoked, or if changes indicative of ischemia can be observed on the ECG. Stress ECG commonly used as an initial screen for coronary artery disease, but is limited by its accuracy rates of only 25-50%. Another commonly used diagnostic test is myocardial perfusion imaging in which a radioactively tagged chemical is injected during stress and is taken up by normally metabolizing cardiac tissue, and then imaged using conventional techniques (PET or SPECT scanning).

The present invention, however, is believed to be advantageous over the known methods of diagnosis in that it is a simple blood test which will offer comparable accuracy at far lower costs and decreased risk and inconvenience to the patient. It is believed that the present invention provides specificity and sensitivity levels that are comparable in accuracy to current diagnostic standards.

It is known that following an ischemic event leading to necrosis, proteins (enzymes, cytoplasmic proteins and structural proteins) are released into the blood. Well known proteins released after such an event include creatine kinase (CK), serum glutamic oxalacetic transaminase (SGOT—also known as ALT and AST—alanine amino transferase and aspartate amino transferase), lactic dehydrogenase (LDH), myoglobin and cardiac troponin (for myocardial necrosis). One well known method of evaluating the occurrence of past heart events is the detection of these proteins in a patient's blood, and in fact the standard of care for diagnosis of Acute Myocardial Infarction is the rise and fall of markers of cardiac necrosis (i.e.: troponin or CK-MB) in the presence of signs and symptoms of cardiac ischemia. The difficulty lies in the diagnosis of ischemia.

U.S. Pat. No. 4,492,753 relates to a method of assessing the risk of future ischemic heart events. However, injured heart tissue releases proteins such as troponin to the bloodstream after both ischemic and non-ischemic events. For instance, patients undergoing non-cardiac surgery may experience perioperative ischemia. Electrocardiograms of these patients show ST-segment shifts with an ischemic cause which are highly correlated with the incidence of postoperative adverse cardiac events. However, ST-segment shifts also occur in the absence of ischemia; therefore, electrocardiogram testing does not distinguish ischemic from non-ischemic events. The present invention provides a means for distinguishing perioperative ischemia from ischemia caused by, among other things, myocardial infarctions and progressive coronary artery disease.

It is an object of the subject invention to provide a diagnostic test that detects a change in a biological molecule by processing a signal produced or altered by the change in the biological molecule, wherein the change relates to the binding of a metal to a portion of the biological molecule.

Another object is to provide a diagnostic test that determines a difference in absorbance and/or fluorescence spectra between plasma, serum, or whole blood samples from ischemic patients and non-ischemic individuals, wherein the samples are first combined with cobalt or another metal.

It is another object of the subject invention to provide an optical assay for detecting a biological condition via detection of a metal binding with a biological sample, wherein there is an increased latitude in the amount of additives such as metal, dye or other reagents added to the biological sample.

Another object of the subject invention is to use data processing techniques such as principal component analysis to identify the features of a spectral output data from an optical assay for differences between ischemic patients and non-ischemic individuals.

It is a further object of the subject invention to reduce the time required to identify a biological condition of a patient, wherein the condition is indicated by an assay that tests for the binding of a metal (e.g., cobalt) to the albumin found in plasma, serum, whole blood or other patient fluid.

It is also an object of the invention to provide a portable apparatus for combining an additive (e.g., a metal) with a sample from a patient and thereby detect/identify a condition related to the health of the patient, wherein the manufacture of the apparatus is reduced in cost due to the fact that additives to be combined with the patient sample need not be measured as precisely as in currently available comparable equipment for detecting or identifying the patient's condition.

It is a further object of the invention to provide a biological assay platform wherein there are a plurality of assay containers with each container having a different metal (and for a fluorescence analysis, a corresponding dye) therein wherein each metal is different and varies according to the biological condition to be detected.

It is an additional object of the invention to provide an apparatus for assaying a patient's condition at the patient's bedside.

A further object of the invention is to measure the rate of change in an optical signal (e.g., absorbance or fluorescence) of an additive combined with a sample from a patient for determining ischemia.

It is an additional object of the invention to continuously or periodically assay small samples of a patient for ischemia analysis, wherein a needle for doing such may have a fiber optic device therein for transmitting and/or receiving light to the sample to be assayed.

### SUMMARY

The present invention is a method and system for detecting a change in a biological system or molecule by processing a signal produced or altered by the change in the biological system or molecule, wherein the change relates to a binding of a metal to portion of the biological system or molecule.

In one embodiment, the present invention is a method and system for determining whether a protein has been altered or damaged by measuring its metal binding capacity. If a protein has the ability to bind metals (or another type of substrate) and the binding site is somehow altered, then it often occurs that the site will either bind less or more to a substrate or ligand. Accordingly, the present invention measures a difference in such binding capacities optically. In particular, any disease state that has an associated alteration of some protein that in turn causes a metal to bind differently than it would in a non-diseased state could be measured using an embodiment of the present invention.

In one particular embodiment of the invention, an improved assay for detecting ischemia is provided, wherein a binding of cobalt ion to albumin is directly measured. In particular, it is believed that cobalt ion binds readily and/or strongly to human serum albumin from patients not having ischemia, and that cobalt ion binds less readily and/or strongly to albumin from patients experiencing ischemia due to an elevated amount damaged binding sites for cobalt on the albumin molecule. This damaged albumin is referred to as Ischemia Modified Albumin, or IMA. Accordingly, one embodiment of the present invention comprises a method for detecting the amount of cobalt bound to albumin directly via absorption spectroscopy in at least the range of 300-450 nm. Moreover, it is believed that spectroscopic signals indicative of the bound cobalt may also be distinguishable in a wider spectral range as well, and in particular, 200-450 nm. In one embodiment for detecting ischemia, a patient serum sample is measured via absorbance spectroscopy with and without cobalt ion, and then the results from the two measurements are subtracted thereby arriving at a difference or differential spectra. This difference spectrum is quantified by, e.g., either a ratio of wavelength intervals or an integration over some spectral interval. In performing various experiments for detecting ischemia in this manner, Applicants have obtained evidence that the spectral measurements and the analysis thereof are indicative of direct cobalt binding to albumin as opposed to detecting free (i.e., unbound) cobalt. Moreover, Applicants have determined that a major advantage of detecting direct cobalt binding to albumin, is that the test is far less sensitive to reagent (e.g., cobalt) concentration than the detection of free (unbound) cobalt. In fact, excess cobalt is believed to be somewhat advantageous in detecting albumin bound cobalt in that the excess cobalt substantially assures that all cobalt binding sites on the albumin will be used.

More generally, it is an aspect of the present invention to combine cobalt or another metal with a sample of plasma, serum or whole blood and determine a difference in spectral absorption between ischemic and non-ischemic patients, wherein the measurements obtained are indicative of the amount of metal bound to albumin within the sample independently of the amount of unbound or free metal (or ions thereof) that may also be in the sample. Additionally, when whole blood is provided as the sample, then the sample can be centrifuged (spun) to obtain the plasma therein, and subsequently in one embodiment, this plasma may be diluted approximately 5 times or more with an appropriate buffer keeping the pH in the range 7.5-8.5, before being optically assayed. Further, when cobalt is the metal used, it has been determined that approximately 15 μL to 40 μL of 1% cobalt solution per approximately 150 μL to 250 μL of plasma is effective for detecting ischemia. More particularly, it has been determined that approximately 25 μL of 1% cobalt solution per approximately 200 μL plasma is effective for detecting ischemia.

In at least some (if not most) embodiments of the invention, a metal compound may be added to the sample thereby causing free metal ions to be introduced into the sample. For example, a 1% cobalt chloride solution in 100 μL of plasma may be used for detecting ischemia, wherein the cobalt chloride provides cobalt ions to the sample. Accordingly, it is to be understood herein that when the term “free metal” or similar terms are used, these terms are intended to mean that unbound metal ions are introduced into a sample.

In another embodiment of the present invention, fluorescence spectroscopy may be performed, wherein a fluorescent dye may be added to a sample of plasma or whole blood or a diluted sample thereof wherein the dye is relatively specific to a particular metal ion, and fluoresces differently in the presence of a free metal (e.g., cobalt, copper or nickel) than in the presence of the metal bound to albumin. In particular, the dyes can indicate the amount of free metal ions or bound metal ions residing in the sample. Moreover, since the dyes contemplated to be used in this embodiment of the invention fluoresce very differently in the presence of free and bound metal, Applicants have discovered that it is unnecessary to precisely calibrate the amount of such a dye to be added to the plasma or blood sample, and as with spectral absorption embodiment above, excess dye is believed to be somewhat advantageous in that this substantially assures that all possible metal bindings by the dye are achieved. Additionally, note that certain dyes to be used fluoresce strongly enough such that the fluorescence can be readily measured in whole blood. It is worth noting that in performing fluorescence spectroscopy according to the present invention, fluorescence signals for the dyes contemplated tend to be quite strong and are therefore quite sensitive to detecting such medical conditions as ischemia. In particular, the following dyes may be used in various embodiments of the invention: Rhodamine, Cumarin and Newport Green.

Applicants have also discovered that it may take time (e.g., 20 minutes) to obtain a steady state (i.e., equilibrium) of bound and unbound metal (e.g., cobalt) within a plasma or whole blood sample. Accordingly, to perform faster assays and for operator convenience, it is an aspect of the present invention to provide the metal ion in the assay container prior to providing the plasma or blood (e.g., during container manufacture). Moreover, to further reduce the assay time, it is an aspect of the present invention to measure a rate of change in the amount of bound metal within a sample at a defined time interval prior to reaction equilibrium instead of the amount of bound metal at equilibrium. This defined time interval can be, for example, any 1-10 minute interval prior to the time of equilibrium. Preferably, the defined time interval is any 1-5 minute or 1-2 minute interval prior to the time of equilibrium. In one embodiment, the interval is selected at 5-15 minutes prior to equilibrium.

The subject invention also comprises a method of optically detecting modifications to the albumin N-terminus using absorbance without the addition of reagents such as metal ions. As is described in the Examples, it has been observed that albumin that has been modified at its N-terminus, as happens during an ischemic event, has a different absorbance spectrum than full length albumin.

In each of the foregoing methods, the optical data from the patient sample obtained is compared to a standard curve or other mathematical model that has been constructed from data collected during clinical trials or other patient studies. The standard curve or mathematical model is used to define the cut-off point between optical data that reflects an ischemic event and that which is indicative of normal or non-ischemic albumin. For example, a set of data from samples collected from non-ischemic people can be used to generate a “normal range”, and the 97^{th} percentile of the upper limit of normal can be defined as the cutoff—any value higher than this is regarded as “ischemic”, and any value lower than this is regarded as “non-ischemic”. Other techniques such as receiver operating characteristic (ROC) curves will be well known to one skilled in the art.

Thus, in a further aspect of the present invention, various signal processing techniques may be used in the analysis of the resulting data obtained from an assay performed according to the present invention. In one embodiment, principal component analysis (PCA) is performed on this resulting data for both data dimension reduction and effectively identifying differences between ischemic and non-ischemic samples using the reduced dimension PCA data set. Principal component analysis (PCA) involves a mathematical procedure that transforms a number of (possibly) correlated variables into a (smaller) number of uncorrelated variables called principal components. The first principal component accounts for as much of the variability in the data as possible, and each succeeding component accounts for as much of the remaining variability as possible. A trivial example of principal component analysis is fitting a straight line to a large and noisy data set plotted in two dimensions. In this case, a large amount of data is reduced to the two variables required to describe the line, and therefore the number of mathematical dimensions required to describe the data set has been reduced enormously.

For example, the absorption spectra (i.e., the resulting data) of a sample is obtained. Subsequently, the resulting data is converted to PCA space to reduce the number of dimensions of the data. In one embodiment, the resulting data may be acquired at a sample rate of approx. 2 measurements per nm, thus giving a spectrum with 300+ components. In PCA the 300+ components are converted to coefficients of basis elements that represent the directions of maximum variation in the resulting data. These basis elements (called principal components or factors) can be ordered from those that describe the most variation to the least. By utilizing only those principal components that describe the most variation, the efficiency of the analysis can be substantially enhanced. Moreover, note that there are several tests to determine how many principal components to include in this subset including the Kaiser Criterion and the Scree test as one skilled in the art will understand. By this conversion, “noise” in the form of the weak components is reduced, and the dimension of the resulting data is reduced as well as the degrees of freedom to be addressed when attempting to detect and/or diagnose a medical condition. Accordingly, the detection/diagnosis is based on this reduced space.

Note that it is also within the scope of the present invention to utilize other signal processing techniques in addition to or as a substitute for PCA such as the following:
a. wavelets (which are often more stable than PCA),
b. Fourier analysis,
c. general factor analysis techniques, and
d. independent component analysis.

There are many other classification techniques that may be used in embodiments of the present invention, including the following:
a. Linear discriminant functions (including piecewise linear);
b. Non-linear discriminant functions (including piecewise non-linear);
c. Cluster techniques to find natural groupings:
   i. Hierarchical,
   ii. Non-hierarchical,
   iii. Density;
d. Mahalanobis distance type metrics from known class means;
e. Multi-dimensional Probability density functions;
f. Neural networks; and
g. Support vector machines.

In yet a further aspect of the present invention, devices are provided for performing the foregoing assays. In one such embodiment, a measurement sample chamber, a reference sample chamber, and a spectrophotometer are provided for measuring spectra or signals in a patient control sample and a test patient sample (the latter containing metal ion and optionally fluorescent dye). These signals or spectra data are transmitted to a computer for determination of the differential spectra or signal, and/or further processing and analysis.

In another embodiment, a device is provided for assaying a (plurality of) patient control(s) and a plurality of patient test samples. Specifically, an assay platform may be provided wherein there is a plurality of assay containers with each container having a different metal (and optionally a fluorescent dye) therein so that the metal may differentially bind with the assay sample depending upon whether the assay indicates one or more medical conditions such as ischemia or non-ischemia. Accordingly, such an optical platform may allow a plurality of such conditions to be diagnosed substantially simultaneously. Additionally, since certain patient treatments may affect the results of some such assays, the assay platform may include redundant assays for the same condition wherein any one of the redundant assays may detect an abnormal medical condition.

Note that some embodiments of the present invention may be performed away from the traditional location of a central hospital laboratory, for example at a patient's bedside. In particular, the invention may be substantially provided in a portable unit that has great flexibility in location, such as adjacent to or attached to a patient's bed. Moreover, such a portable embodiment may include a hypodermic needle having a fiber optic device therein for transmitting and/or receiving light to a sample to be assayed. Thus, assays may be performed continuously or periodically on small samples from a patient.

Other advantages and benefits of the present invention will become apparent from the accompanying drawings and Detailed Description herein below.

All references cited are incorporated by reference herein in their entirety.

### BRIEF DESCRIPTION OF THE DRAWINGS

FIG. 1 shows an embodiment of the apparatus for the present invention that obtains and processes optical signal data for detecting ischemia.

FIG. 2 is an alternative embodiment to the apparatus of FIG. 1 for performing the present invention.

FIG. 3 shows a graph of optical data distinctions between ischemic patients and non-ischemic patients when plasma samples for both types of patients are combined with cobalt.

FIG. 4 shows the graphical results of further tests performed to determine the buffer strength effect on cobalt binding to purified albumin.

FIG. 5 shows the graphical results of further tests performed to determine the pH effect on cobalt binding to purified albumin.

FIG. 6 shows the graphical results of further tests performed to illustrate that normal human serum displays the cobalt binding effect, and that the effect saturates with increased cobalt.

FIGS. 7, 8 and 9 show the graphical results of further tests performed to illustrate that there can be approximately 90% recovery of the cobalt binding even in the presence of very high concentration of a (over 1000× of what would be expected in a biological sample) chelator (both citrate and EDTA).

FIGS. 10 and 11 show the graphical results of further tests performed to determine whether albumin precipitates with high cobalt concentrations by comparing centrifuged and un-centrifuged samples.

FIG. 12 shows that the direct cobalt binding with albumin is not adversely affected by chelators.

FIG. 13 shows the change in absorbance when cobalt (at pH 8) is added to purified albumin.

FIG. 14 illustrates the results obtained from analyzing signal data of both ischemic patients and non-ischemic patients using PCA.

FIGS. 15A and B illustrate the effect on absorbance when cobalt ion is added to albumin N-terminal models.

FIGS. 16 and 17 illustrate how the absorbance of Co-albumin mixtures can be used to quantify the amount of N-terminally modified albumin.

FIG. 18 illustrates how the absorbance of albumin (without metal reagent) can be used to quantify the amount of N-terminally modified albumin.

### DETAILED DESCRIPTION

According to the invention, a method is provided for diagnosing an ischemic event by obtaining a first and second patient sample which include albumin (e.g.: whole blood, plasma, or serum), adding to the first patient sample a metal ion that binds to the albumin, conducting optical analyses on the first and second samples to generate optical signals or spectra, measuring the amount of metal bound to the albumin by comparing the signals or spectra of the first and second samples to generate a differential signal or spectra, and comparing the differential signal or spectra to a standard curve or mathematical model that correlates the differential signal or spectra to an amount of metal bound to albumin, whereby an ischemic event can be diagnosed if the measurement of metal bound to albumin is below a defined value.

Preferably, the first and second patient samples are derived from the same bodily fluid, e.g., blood, serum, plasma, saliva, cerebro-spinal fluid, and the like. Typically, the first and second patient samples are provided by dividing an original patient sample of a bodily fluid with a suitable buffer to maintain the pH within a specific range. The choice of buffer and the concentration to be used will be determined by the precise configuration of the test apparatus, but applicants have found that ammonium acetate buffer with a pH of approximately 8 givers satisfactory results in the prototype apparatus.

The metal ion used in the assay can be any metal ion that binds to the albumin, including transition metal ions of Groups 1b-7b or 8 of the Periodic Table of the Elements, or a metal selected from the group consisting of V, As, Co, Cu Sb, Cr, Mo, Mn, Ba, Zn, Ni, Hg, Cd, Fe, Pb, Au and Ag. Preferably the metal ion is a cobalt ion. The metal ion is believed to bind to the N-terminus of albumin that has not been damaged during an ischemic event. Albumin that has been altered at its N-terminus during an ischemic event is unable to bind to cobalt ion or other metals. A detailed description of the molecular events underlying an ischemic event and the resulting damage to albumin is set forth in PCT/US99/22905, filed Oct. 1, 1999, which is incorporated by reference herein in its entirety.

The standard curve is generated by plotting differential optical spectra or signal data against actual metal bound to albumin for samples from normal individuals and patients diagnosed as ischemic by other methods known in the art, such as the ACB™ Test (Ischemia Technologies, Inc., Denver, Colo.), and other tests for ischemia such as electrocardiogram and myocardial perfusion imaging. Actual metal bound to the albumin in the samples can be determined by, for example, purifying the albumin and assaying for metal using methods known in the art (e.g., absorption chromatography and atomic absorption).

As in all diagnostic tests, a “normal range” study should be performed in each laboratory performing the test. At some point on the standard curve, a cut-off value that corresponds to an ischemic event is defined. Below the cut-off point, there is so much ischemia modified albumin that metal binding to the albumin is diminished to the point that the patient is considered to be undergoing an ischemic event. Thus, in one embodiment of the invention described in this application, “more ischemic” values will be lower numbers because of less absorbance of the cobalt N-terminus complex, and “less ischemic” will be higher because of more absorbance due to higher concentrations of the cobalt N-terminus complex. Obviously, a mathematical transformation could be performed to change the reported values so that “more ischemic” is a higher number than “less ischemic”, and appropriate units assigned to the reported value.

While standard curves can suffice to correlate optical data with metal-albumin binding and clinical diagnosis, mathematical models derived from other data processing techniques are also available. As discussed above, PCA is useful in reducing noise and identifying patterns in the spectral data. This permits the elucidation of a mathematical model which permits sensitive and specific diagnosis of an ischemic event. Sensitivity is typically regarded as the number of true positive results divided by the number of true positive plus false negative results; it is the probability that a person having a condition will be correctly identified by a clinical test for that condition. Specificity is the probability that a person not having a condition will be correctly identified by a clinical test for the condition; it is calculated by dividing the number of true negative results by the number of true negative plus false positive results.

The optical analysis conducted on the patient samples can be absorbance spectroscopy or fluorescence spectroscopy. The absorbance spectroscopy is preferably conducted in the range of 200-450 nm, and more preferably in the range of 300-450 nm or 305-350 nm.

Where the optical analyses is fluorescence spectroscopy (and a first and second patient sample are used), a fluorescent dye is added with the metal ion to the first patient sample. The dye binds to the metal ion and the fluorescent signal changes as a function of whether the metal ion is unbound or bound to the albumin.

In another embodiment, the invention provides a method for diagnosing an ischemic event by adding a metal ion and a fluorescent dye to a patient sample comprising albumin, whereby the dye binds to the metal ion which in turn may bind to the albumin. In this embodiment, there is only a single patient sample. Again, the fluorescent signal changes as a function of whether the metal ion is unbound or bound to the albumin. The fluorescent signal of the sample is measured and compared to a standard curve or mathematical model that correlates the fluorescent signal to an amount of metal ion bound to albumin. If the amount of metal ion bound to albumin is below a defined cut-off value, an ischemic event may be diagnosed. The metal ion and fluorescent dye may be added separately or as a conjugate. While the metal ion may be any metal that binds to unmodified albumin, it is preferably a cobalt ion. The fluorescent dye is preferably Cumarin, Rhodamine or Newport Green.

In another embodiment of the invention, a method is provided for rapidly diagnosing an ischemic event by evaluating the rate of change of metal binding to albumin as indicated by absorbance measurements. A first and second patient sample which include albumin (e.g.: whole blood, plasma, or serum), are obtained, and a metal ion is added to the first patient sample. The metal ion binds to the albumin in a reaction that reaches equilibrium at a predetermined time. For a defined time interval prior to achievement of equilibrium, optical analyses of the first and second samples are conducted, and signals or spectra for each sample at selected time points in the defined time interval are obtained. The rate of change of amount of metal bound to the albumin is measured over the defined time interval by comparing the signals or spectra of the first and second samples for each time point to generate differential signals or spectra for each time point in the time interval. Then the rate of change in the differential signals or spectra over the time interval is calculated, and the rate of change of differential signal or spectra is compared to a standard curve or mathematical model that correlates rate of change with projected metal bound to albumin at equilibrium, whereby an ischemic event may be diagnosed if the projected amount of metal bound to albumin is below a defined value.

In another embodiment, the subject invention provides a method of rapidly diagnosing an ischemic event by evaluating rate of change of metal binding to albumin as indicated by fluorescent spectra or signals. A metal ion and a fluorescent dye are added to a patient sample comprising albumin, whereby the dye binds to the metal ion which binds to the albumin in a reaction that reaches equilibrium at a predetermined time. The fluorescent dye's signal changes as a function of whether the metal ion is unbound or bound to the albumin. The rate of change of metal bound to the albumin is measured by measuring the fluorescent signal of the sample at selected time points over a time interval that is prior to achievement of equilibrium, calculating the rate of change of the fluorescent signal over the time interval, and comparing the rate of change of the fluorescent signal to a standard curve or mathematical model that correlates the rate of change in fluorescent spectra or signal to a projected amount of metal ion bound to albumin at equilibrium. If the measured rate of change of metal ion bound to albumin is below a defined value, an ischemic event may be diagnosed.

The subject invention further comprises a method for diagnosing an ischemic event by measuring the N-terminally modified portion of albumin in a patient sample by measuring absorbance of the sample, and comparing the absorbance to a standard curve or mathematical model that correlates the absorbance to a ratio of modified to unmodified albumin. An ischemic event may be diagnosed if the ratio is below a defined value. In this embodiment, no reagent (metal ion) is added to the patient sample. The patient sample can be whole blood, serum or plasma provided in a sample container. In the alternative, the patient sample can be the whole blood in the patient's blood vessel, with the absorbance being measured with a spectral probe placed in the blood vessel.

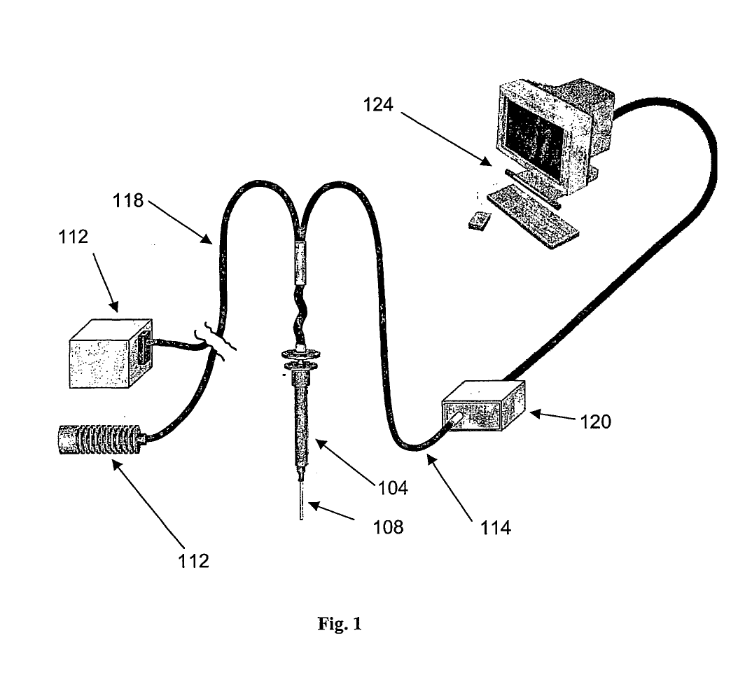

One embodiment of the medical assay system and method of the present invention is illustrated in FIG. 1 for optically detecting ischemia. A spectral probe 104 is provided for insertion of its tip 108 into a sample to be assayed. In particular, the spectral probe 104 and its tip 108 can be further described as follows. The spectral probe is an apparatus that draws in a very small volume of a sample (˜3 μL) and passes the light through the sample liquid, reflects the light back to a collection optical fiber 114 that is attached to the spectrometer 120.

Attached to the spectral probe 104 via a supply optical fiber 118 is at least one of a laser 112 and a broadband light source 116 for supplying light, e.g., in at least the range of 300-450 nm. More particularly, the laser 112 and the broadband light source 116 can be further described as follows. The broadband light source 116 is any source of light that is capable of creating a continuum of wavelengths in some interval (or a decent approximation to a continuum). A laser light is any source that creates a very narrow or discrete wavelength.

Attached to the spectral probe 104 via collection optical fiber 114 is the spectrophotometer 120 for receiving output light from the spectral probe 104. The spectrophotometer 120 includes the functionality for quantifying each frequency of light input thereto.

Digital data corresponding to the light received at the spectrophotometer 120 is output to a computer 124 for signal processing according to the present invention. In particular, the computer 124 may perform PCA analysis (or another signal processing technique as discussed herein) as well as visually display the results for detecting ischemia and/or various graphical characterizations of data derived from the output of the spectrophotometer 120.

The embodiment of FIG. 1 can be modified to provide a plurality of spectral probes 104 that are dipped manually or in an automated fashion into a plurality of sample tubes in a sample array (not shown). Each spectral probe 104 provides data to the spectrophotometer 120 and computer 124, for multiple analysis of a plurality of samples from a single individual or multiple individuals.

FIG. 1 shows an embodiment of the apparatus where the tip of the probe 108 as described is placed into a container holding the patient sample—in other words, the device is configured as an in vitro diagnostic device. In a modification to this embodiment, the probe is made small enough to be placed into an indwelling arterial or venous line in a patient to allow semi-continuous monitoring of the ischemic state of a patient. In this embodiment, additional means are necessary to draw in a sample of the patient's circulating blood, allow mixture with an excess of cobalt, and then spectral measurement of the resulting solution using the same apparatus. Alternatively, the probe may merely measure the albumin absorbance spectra without the metal reagent, so as to detect relative concentrations of unmodified and N-terminally modified albumin.

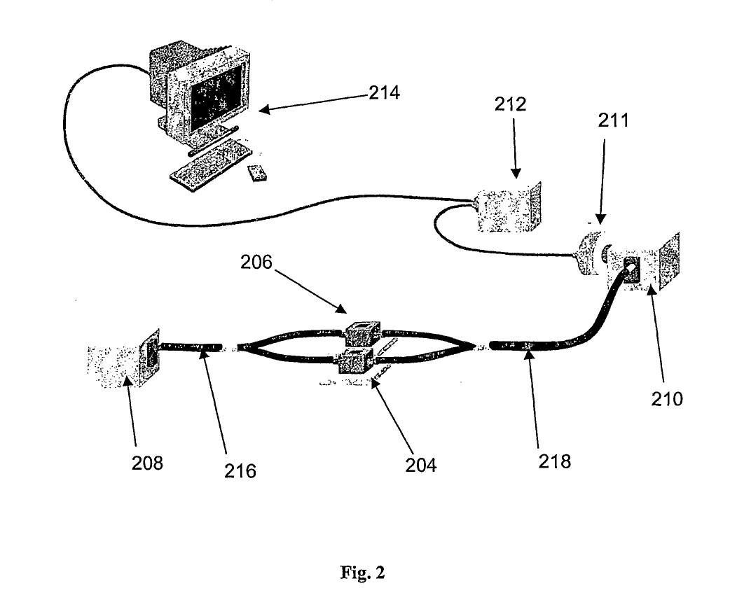

FIG. 2 shows an alternative embodiment of the apparatus to perform the assays of the present invention. This embodiment includes a reference sample chamber 204 for measuring a control sample, a measurement sample chamber 206 for measuring the actual sample to be assayed, a broadband light source 208 for generating a continuum of light in some interval, and a supply optical fiber 216 for delivery of the broadband light to the reference and measurement chambers 206, 208. The light source 208 may be a deuterium tungsten lamp. Also provided is a collection optical fiber 218 for conveyance of the transmitted light to a spectrometer 210 for quantifying the amount of each wavelength received. The spectrometer 210 may be an Acton Instrument 150 commercially available from Princeton Instruments. The spectrometer 210 is observed by a camera 211, which could for example be a CCD camera, which supplies optical data to the controller 212 and the spectrometer 210 also receives controlling signals from the controller 212. The controller 212 provides analog to digital signal conversion as well as receives controlling signal from the computer 214, which requests spectral data in a particular optical range and with a specified exposure. The computer 214 performs the signal processing analysis, stores results, displays and processes data, and optionally performs certain reliability checks on the other components.

The subject invention further provides an instrument for detecting a medical condition, which comprises a spectral probe having a tip for insertion into a patient fluid sample and for receiving spectral light from the patient fluid sample. A spectrophotometer is coupled to said spectral probe, and quantifies each frequency of spectral light received from the spectral probe; it also outputs a signal representative of the quantity of each frequency of spectral light. The instrument also has a computer with an input coupled to receive the signal from the spectrophotometer. The computer also has a memory for storing a model representing spectral light data obtained from a first set of patients known to have the medical condition and a second set of individuals known to not have the medical condition, whereby the model includes a value identified with a high probability of the presence of the medical condition. The computer also has a processor programmed to execute instructions for comparing the quantity of each frequency of spectral light from the patient with corresponding data in the stored model; and determining whether the quantity of each frequency of spectral light is indicative of the presence of the medical condition in the patient. Finally, the computer has an output to provide the determination to the user.

The invention also comprises a method for providing an instrument for diagnosing a medical condition in a patient. The method involves obtaining a control fluid sample from a first plurality of control individuals known to have the medical condition, and obtaining a control fluid sample from a second plurality of control individuals known to not have the medical condition. Each control sample is divided into first and second portions, and the first portion is combined with free metal ions. Then, both the first and second portions of each control fluid sample are irradiated with light, and absorbance values for the first and second portions of each control fluid sample are determined. Next, a differential absorbance value is obtained from the first and second portions of each control fluid sample. A principal component analysis (PCA) model of the obtained differential absorbance values is generated. The PCA model includes a value indicative of the presence of the medical condition. This PCA model is stored in a computer readable format. Computer executable instructions are provided for determining a differential absorbance value from first and second portions of a patient fluid sample (the first portion having been combined with free metal ions), and comparing the differential value with the stored PCA model. Next, the computer determines whether the differential absorbance value of the patient fluid sample is indicative of the presence of the medical condition.

### EXAMPLES

### Example 1

Correlation of Absorbance Spectroscopy Differential (ASD) to Clinical Diagnosis of Ischemia

Using the device of FIG. 1, spectra of plasma from a total of fifteen individuals with and without clinical ischemia were analyzed to determine if ischemia could be detected spectroscopically, and in particular, whether ischemia induced damage to albumin could be detected. The plasma samples used are characterized in Table 1. For each sample, 100 ì L of plasma ±25 ì L of CoCl₂.6H₂O 0.1% were reacted for 2-5 minutes and then subjected to analysis by the apparatus illustrated in FIG. 1. Spectra from 200-350 nm were obtained with and without cobalt (e.g., CoCl₂). Differences in the resulting output spectrums were analyzed by performing an integration of the graph of the differential spectra. However prior to performing the integration, the differential spectra obtained from the differences were shifted so that the baseline in the deep UV (200-300 nm) was zero. Subsequently, each differential spectra was integrated from 305-350 nm. The resulting integral value was used to determine whether a correlation with ischemia could be obtained.

Table 1 shows the summary of the results from 7 patients with several samples from each patient taken at varying time points during hospitalization. In this table, the column “Sample Label” is the index number of the sample tested, and if insufficient sample was available to test, it is entered as “N/S”. The column labeled “Ischemia Test” is an automated adaptation of result of the manual assay for cobalt binding as described in Bar-Or, D.et al. (2000) J. Emerg. Med. 19:4. The automated assay is substantially in the form described in the paper Christenson R. L., et al., (2001) Clinical Chemistry 47(3):464-470.

The cutoff of “ischemic” using the automated modified assay is any sample with a test result greater than 80 U/mL. The column labeled “TnI” is the result of an assay for Troponin I where the cutoff for diagnosis of Acute Myocardial Infarction is taken as 1.5 ng/mL (according to the manufacturer's labeling), and symbol “+” is entered before the result if it above the cutoff, and therefore indicative that the patient had ischemia at some time prior to the sample being taken. The column labeled “Adjusted 305-350 Integral” is the computation of the spectrum from 305-350 nm (no result is entered if insufficient sample was available).

TABLE 1
University of Tennessee Knoxville, Ischemia Samples

| | Sample | | | Adjusted 305-350 |
|---|---|---|---|---|
| Patient | Labels | Ischemia Test | TnI | Integral |
| Patient 1 | 14 | −50 | −0.3 | −4.78 |
| | 12 | −64 | −0.3 | 61.95 |
| | 13 | +/−71 | −0.3 | 42.13 |
| Patient 2 | 11 | +/−75 | +2.4 | 9.28 |
| | N/S | +89 | +37.3 | |
| | N/S | −64 | +19.9 | |
| | 10 | +90 | +13 | 55.01 |
| Patient 3 | 7 | −48 | −0.3 | 23.31 |
| | 3 | −58 | −0.3 | −10.29 |
| | N/S | −45 | −0.3 | |
| Patient 4 | 2 | −60 | +5.8 | 125.88 |
| | 15 | +81 | +88 | 44.23 |
| | 8 | +/−75 | +108 | 36.78 |
| Patient 5 | 6 | −62 | −0.3 | 8.61 |
| | N/S | +84 | −0.3 | |
| | 4 | −65 | −0.3 | 6.32 |
| Patient 6 | 5 | −59 | +/−1.5 | 53.00 |
| | N/S | +105 | +5.5 | |
| | 1 | +106 | +5.5 | −4.80 |
| Patient 7 | N/S | +92 | −0.3 | |
| | 9 | +86 | +2.2 | 39.19 |
| | N/S | +92 | +4.6 | |

The sensitivity and specificity of the Absorbance Spectroscopy Differential (ASD) calculated from the data in Table 1 is presented in Table 2. Accordingly, Table 2 illustrates that even when relatively simple signal processing analysis is performed, there is substantial correlation in identifying patients who have an acute cardiac event characterized by elevation of troponin I (e.g.: Acute Myocardial Infarction, where ischemia precedes necrosis), or patients undergoing ischemia as characterized by an elevation in the ischemia test.

TABLE 2
ASD Performance vs TnI and Ischemia Test

| | |
|---|---|
| Sensitivity vs. TnI Only | 87.50% |
| Specificity vs. TnI Only | 57.14% |
| Accuracy vs. TnI Only | 73.33% |
| Sensitivity vs. Either TnI or Ischemia Test | 88.89% |
| Specificity vs. Either TnI or Ischemia Test | 66.67% |
| Accuracy vs. Either TnI or Ischemia Test | 80.00% |

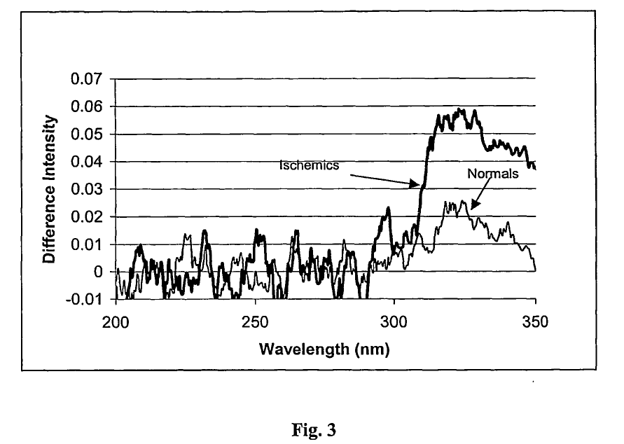

Accordingly, ASD appears to distinguish between ischemic and non-ischemic patients with relatively simple signal processing analysis performed. In particular, as illustrated in FIG. 3 the enhanced amplitude of the wave at 310-350 appears to correlate to the presence of ischemia. Additionally, note that Table 3: Calculations and Statistical Summary for Example 1, provides further detail as to the computations performed in obtaining FIG. 3. In the table, integral values over 200-300 nm and 305-350 nm are set forth for individuals 1-15; individuals 1, 2, 5, 8, 9, 10, 11, 13 and 15 are patients diagnosed with ischemia, and patients 3, 4, 6, 7, 12 and 14 are diagnosed to not have ischemia, according to clinical criteria. “TP” is a 1 if the data is a True Positive (i.e.: the ischemia diagnosis is positive, and the adjusted 305-350 nm calculation is positive with a cutoff of 9 (i.e., >9 is taken as “ischemic”). Similarly, TN is true negative, FP is False Positive, and FN is False Negative. These data are used to calculate the sensitivity and specificity of the test using conventional statistical techniques. The mean and standard deviation of the 305-350 nM are also calculated for both the ischemic and non-ischemic population, and a standard statistical calculation shows that the populations are different at the p=0.08 level (i.e., there is less than 8% chance that the two populations are the same). Accordingly, it is believed that more sophisticated signal processing techniques such as PCA and others discussed herein will yield a better detection of ischemia.

TABLE 3
Calculations and Statistical Summary for Example 1

| | Non-Ischemic Patients | | | | | | Ischemic Patients | | | | | | | | |
|---|---|---|---|---|---|---|---|---|---|---|---|---|---|---|---|
| | 3 | 4 | 6 | 7 | 12 | 14 | 1 | 2 | 5 | 8 | 9 | 10 | 11 | 13 | 15 |
| Mean | 0.0537 | −0.022 | 0.0036 | −0.042 | −0.011 | −0.012 | 0.0777 | −0.248 | 0.1243 | 0.032 | 0.1773 | 0.0197 | 0.1191 | 0.0035 | 0.0368 |
| 200 | | | | | | | | | | | | | | | |
| to | | | | | | | | | | | | | | | |
| 300 | | | | | | | | | | | | | | | |
| Ad- | −10.29 | 6.32 | 8.61 | 23.31 | 61.95 | −4.78 | −4.80 | 125.88 | 53.00 | 36.78 | 39.19 | 55.01 | 9.28 | 42.13 | 44.23 |
| justed | | | | | | | | | | | | | | | |
| 305- | | | | | | | | | | | | | | | |
| 350 | | | | | | | | | | | | | | | |
| TP | | | | | | | 0 | 1 | 1 | 1 | 1 | 1 | 1 | 1 | 1 |
| TN | 1 | 1 | 1 | 0 | 0 | 1 | | | | | | | | | |
| FN | | | | | | | 1 | 0 | 0 | 0 | 0 | 0 | 0 | 0 | 0 |
| FP | 0 | 0 | 0 | 1 | 1 | 0 | | | | | | | | | |
| Mean | 14.19 | | | | | | 44.52 | | | | | | | | |
| Stan- | 26.14 | | | | | | 36.35 | | | | | | | | |
| dard | | | | | | | | | | | | | | | |
| Devi- | | | | | | | | | | | | | | | |
| ation | | | | | | | | | | | | | | | |
| p-test | 0.08 | | | | | | | | | | | | | | |

### Example 2

Effect of Buffer Strength on Co Binding to Albumin

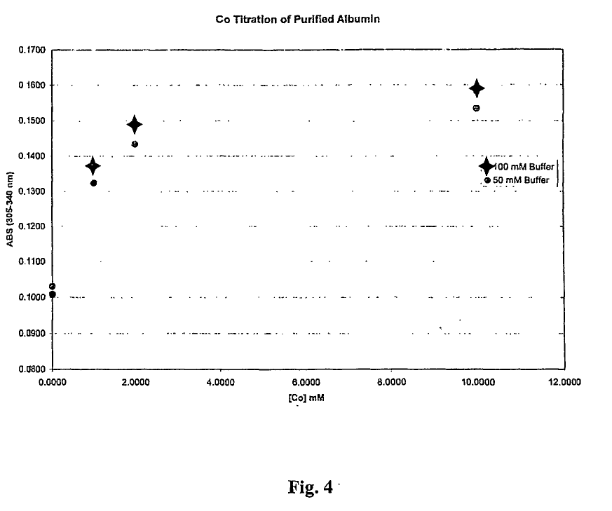

Further experiments were performed to determine the buffer strength effect on cobalt binding to purified albumin. In this experiment, the buffer used was ammonium acetate. In particular, the test provides an indication as to whether the buffer strength had a significant effect on the purified albumin cobalt titration curve. The buffers used were 50 and 100 mM ammonium acetate pH 7.5. The titration curves shown in FIG. 4 did not appear to differ significantly both in the rate at which the Absorbance (ABS) increased and the final ABS level for the two buffer concentrations tested.

### Example 3

Effect of Buffer pH on Binding of Co to Albumin

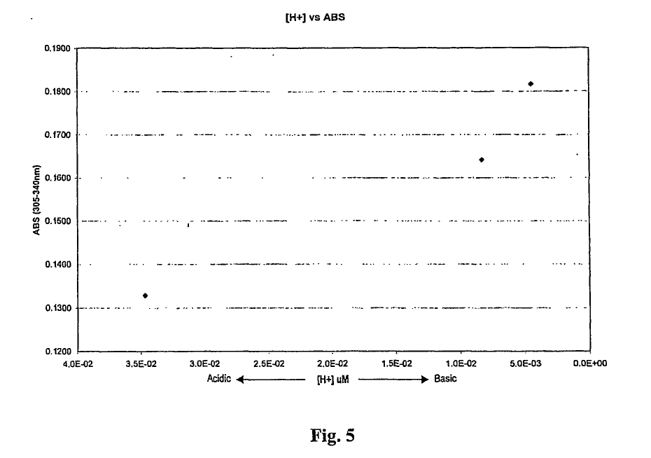

FIG. 5 shows the graphical results of further tests performed to determine the pH effect on cobalt binding to purified albumin. In particular, the test provides an indication as to whether buffer pH had a significant role in the final ABS level. The buffers used were all ammonium acetate with 1 mM CoCl₂. The cobalt level was chosen since it fell well below the saturation level. The pH's used were 7.46, 8.08, and 8.35. It was concluded that buffer pH plays a significant role in cobalt binding. The more alkaline buffers bound more cobalt and had larger increase in ABS. This experiment suggests that raising the pH of the buffers is as important as having a large excess of cobalt.

### Example 4

Absorbance Measurement Reflects Co Ion Bound to Albumin

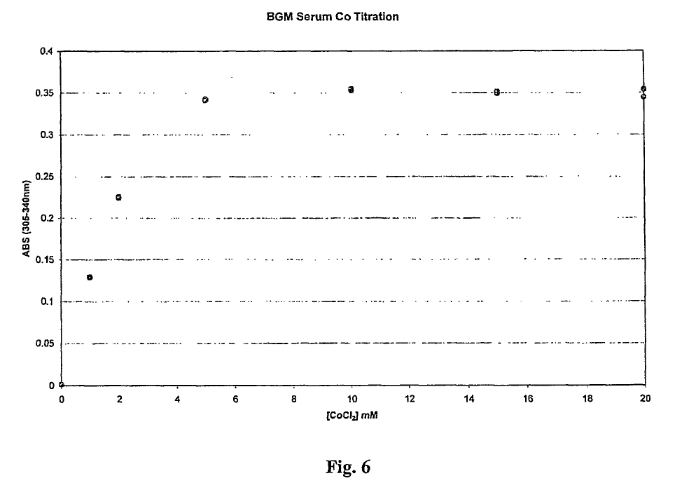

Further tests were performed to illustrate that normal human serum displays the cobalt binding effect, and that the effect saturates with increased cobalt. This is evidence for direct cobalt binding measurement, since if the effect were only measuring total cobalt or free cobalt, then the effect would continue to increase with increased cobalt concentration, whereas if the effect is measuring cobalt bound to albumin, then the ABS would reach a maximum when all the albumin was bound with cobalt. In particular, the test was performed with human serum titrated with CoCl₂ in ammonium acetate buffer at pH 7.5. Concentrations of CoCl₂ used in the titration are 0, 1, 2, 5, 10, 15 and 20 mM. FIG. 6 shows that the integral ABS over 305-340 nm for Co titration of human serum saturates above approximately 5 mM. Therefore the effect is a measure of cobalt bound to albumin only.

### Example 5

Effect of Chelators on Availability of Co Ion for Binding to Albumin

When blood samples are collected from a patient, they are often collected in a tube with a chemical to prevent clotting, to allow centrifugation of the sample to yield the plasma. The chemicals in use to prevent clotting include heparin, EDTA, oxalate and citrate. EDTA, oxalate and citrate are chelators (i.e.: they bind metal ions), therefore there is a potential that the presence of these chemicals might interfere with the test to detect metal ion binding to the N-terminus of albumin. Experiments were performed to investigate whether or not plasma samples collected in EDTA or citrate tubes might give erroneous results.

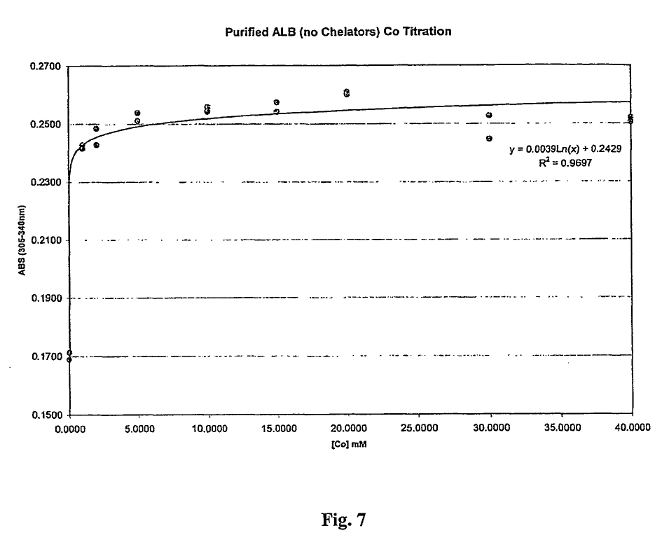

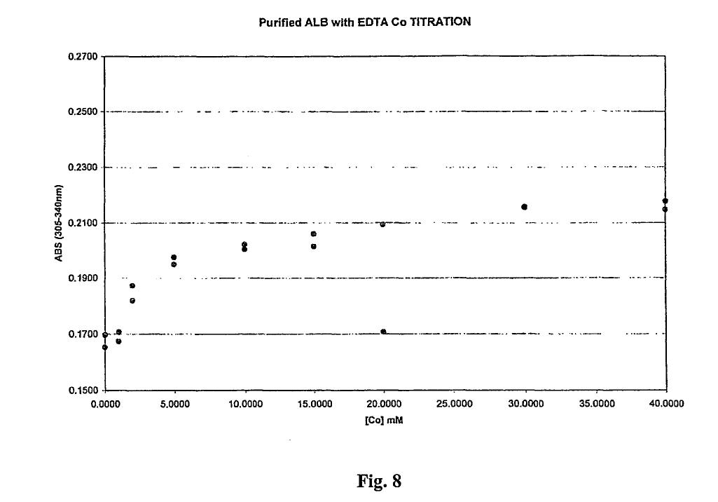

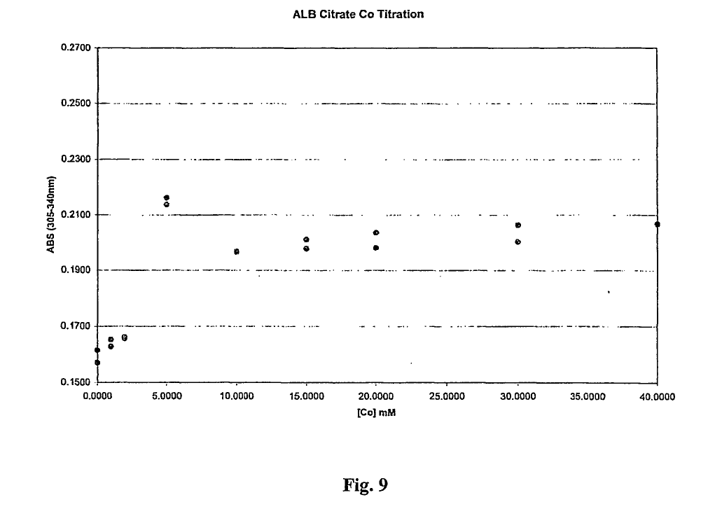

FIGS. 7, 8 and 9 show the graphical results of tests performed to illustrate that there can be approximately 90% recovery of the binding of Co ion to the albumin even in the presence of very high concentration (over 1000× of what would be expected in a biological sample) of a chelator (both citrate and EDTA). FIG. 7 shows the integral ABS curve (305-340 nm) for purified albumin (no chelators) with increasing concentrations of Co. FIG. 8 shows the integral ABS curve (305-340 nm) for purified albumin with EDTA and increasing concentrations of Co ion. FIG. 9 shows the ABS (305-340) for purified albumin with citrate, and increasing concentrations (0-40 mM) of Co ion. Similar experiments were performed in which the concentration of chelator (EDTA or citrate) was varied, which also showed minimal effect on the ABS due to albumin cobalt binding.

Note that a Co ion binding recovery of 90% is important in that the chelators may come in contact with a patient sample (e.g.: from the sample collection tube) before an assay according to the present invention is administered, thereby creating a deleterious effect on the outcome of the assay. For example, in embodiments of the invention that measure free cobalt, such an assay may yield a falsely normal result since the cobalt may bind to the chelator thereby causing less free cobalt. In embodiments of the invention that measure cobalt bound to albumin, sequestration of Co ion by chelators may show up as a decrease in the cobalt binding (less available to bind to albumin) and therefore a falsely abnormal test result. Accordingly, FIGS. 7, 8 and 9 show that when measuring cobalt bound to albumin, a large excess of cobalt can saturate the chelator and therefore provide sufficient available free cobalt for binding to the albumin. Thus, this large excess of free cobalt causes the assay results to be nearer the results expected had the chelator not been present.

### Example 6

High Co Ion Concentration Does Not Precipitate Albumin.

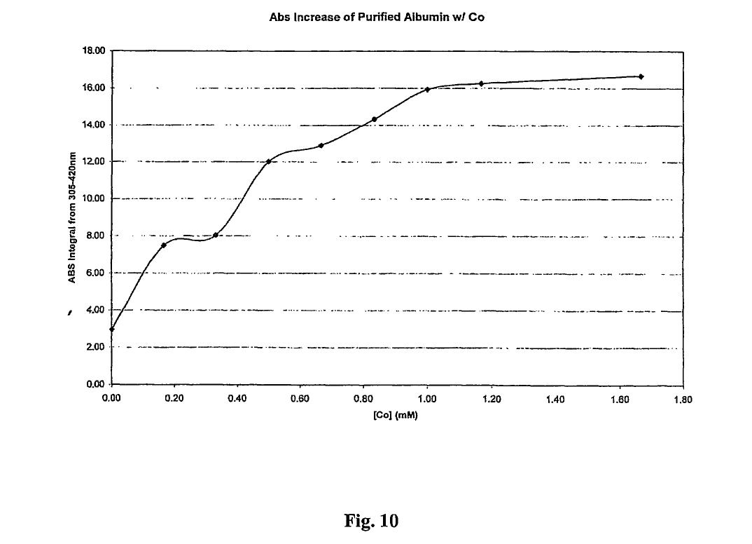

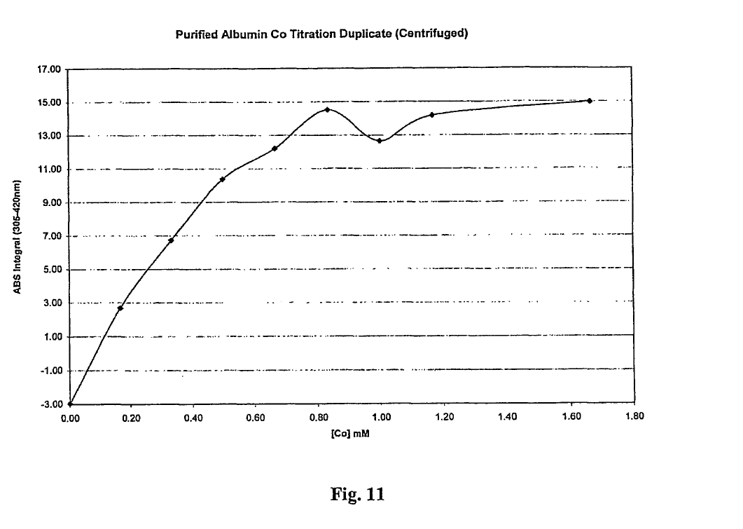

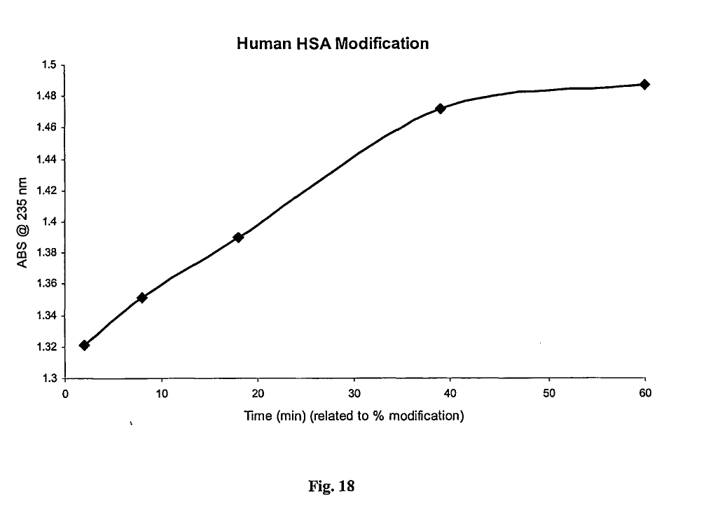

One of the concerns about adding high concentrations of cobalt to a sample is that it may cause precipitation of the albumin, which would adversely affect the results of the test. An experiment was performed in which different amounts of cobalt were added to a preparation of pure albumin. The preparation was then measured to determine the ABS integral value, with and without centrifugation. If high concentrations of cobalt cause precipitation of albumin, then it would be expected to see a different value in ABS integral for the centrifuged and non-centrifuged samples. FIGS. 18 and 20 show ABS integral values (305-420 nm) for Co bound albumin, without centrifugation and with centrifugation, respectively. From the similarities of FIGS. 10 and 11, it appears that precipitation does not occur, and that cobalt binding measurement is not affected by high cobalt concentrations.

### Example 7

Effect of EDTA Chelator on Cobalt Binding to Albumin

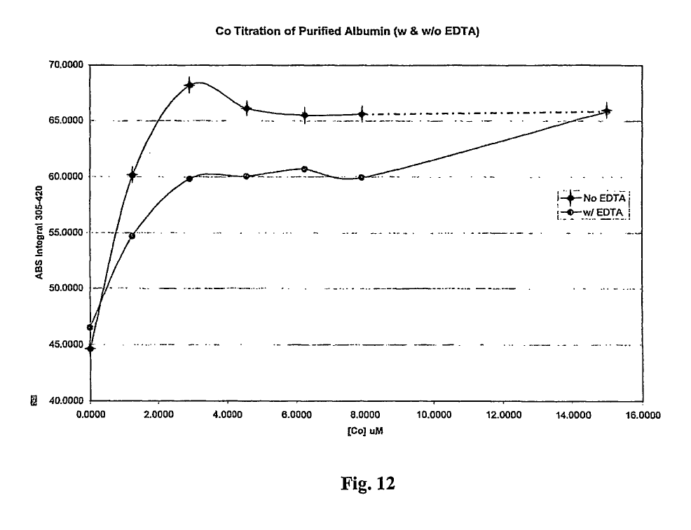

An experiment was performed to determine if the presence of chelators (in this case EDTA) affects the direct binding of cobalt to albumin as measured by the ABS integral. A preparation of purified albumin was spiked with EDTA, and the measurements of the ABS integral were made with increasing amounts of cobalt added to the preparation. FIG. 12 shows that the direct cobalt binding with albumin is not significantly adversely affected by chelators.

### Example 8

ABS Spectra of the Albumin Cobalt and Co-Albumin Complex

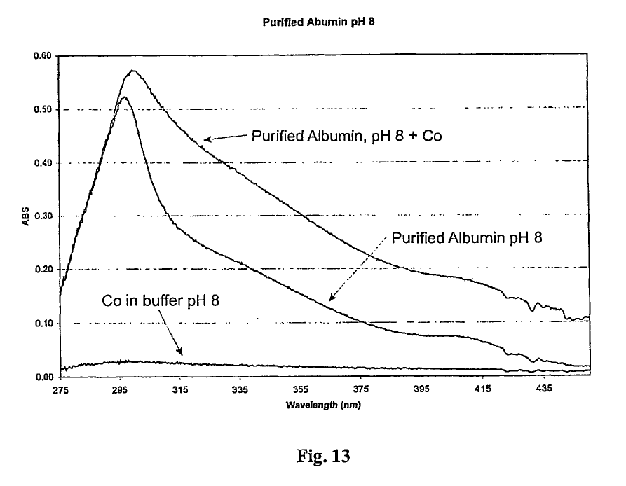

The question arises as to whether the value of ABS integral is due to differences in Co concentration, or Co-Albumin complex. An experiment was performed in which ABS integral was measured in a solution of Co alone, albumin alone, and Co added to Albumin. FIG. 13 shows the change in ABS integral when cobalt (at pH 8) is added to purified albumin. In particular, the graph of FIG. 13 shows a change in the optical absorbance of albumin with the addition of cobalt, which shows that the value of ABS is not due to the cobalt alone, but rather the combination of Co and albumin.

### Example 9

Principal Component Analysis (PCA) of Ischemic and Non-Ischemic Data

PCA is a linear model which transforms the original variables of a spectrum (data set) into a smaller set of linear combinations of the original variables called principal components that account for most of the variance of the original data set. Principal component analysis is described in Dillon W. R., Goldstein M., Multivariate Analysis: Methods and Applications, John Wiley and Sons, 1984, pp. 23-52, the disclosure of which is expressly incorporated herein by reference. PCA provides a novel approach of condensing all the spectral information into a few manageable components, with minimal information loss. Furthermore, each principal component can be easily related to the original emission spectrum, thus providing insight into diagnostically useful emission variables.

PCA is a pattern recognition technique used to classify a set of analyzed samples. PCA defines axes in space that describe the major sources of variance in measurements taken on the samples, contained in a matrix of independent variables R. The new axes are called the principal components (PCs). The coordinates of the samples in the rotated space are called the scores. The spatial orientation of the analyzed samples can be examined visually using scores vs. scores plots in the two dimensional planes defined by the PCs. In these projections, clusters of samples often appear, indicating that these samples had a similar covariance for the measured variables and may be inherently similar in a chemical, physical, etc., sense.

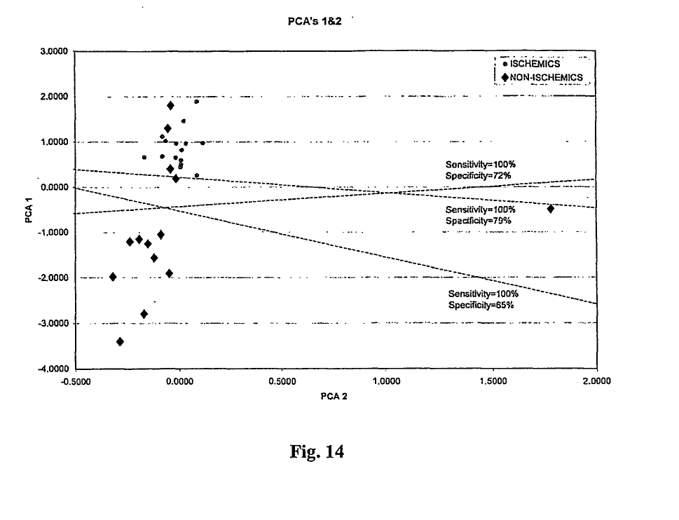

PCA results were obtained from analyzing signal data of both ischemic patients and non-ischemic individuals. Note that the signal data used here was obtained from an embodiment substantially as in FIG. 1. It was found that the first two principal components (referred to as PCA1 and PCA2) yielded most of the information in the data set. In particular, FIG. 14 shows the results of ischemic and non ischemic patients plotted in PCA space, thereby illustrating that two distinct clusters or groups are capable of being derived from the use of these two components. Additionally, FIG. 14 shows several classification schemes for classifying an outlier located at the approximate coordinates of (1.7, −0.5), and the different sensitivity and specificity available with each of the classification schemes.

One embodiment of the software used to compute the PCA principal components described hereinabove is set forth in Appendix A, which is incorporated herein by reference. This program is written in the mathematical system commercially known as Mathematica produced by Wolfram Research, Inc. at 100 Trade Center Drive, Champaign, Ill. 61820-7237, USA.

### Example 10

Effect on ABS of Co Ion Binding to Albumin N-Terminal Models

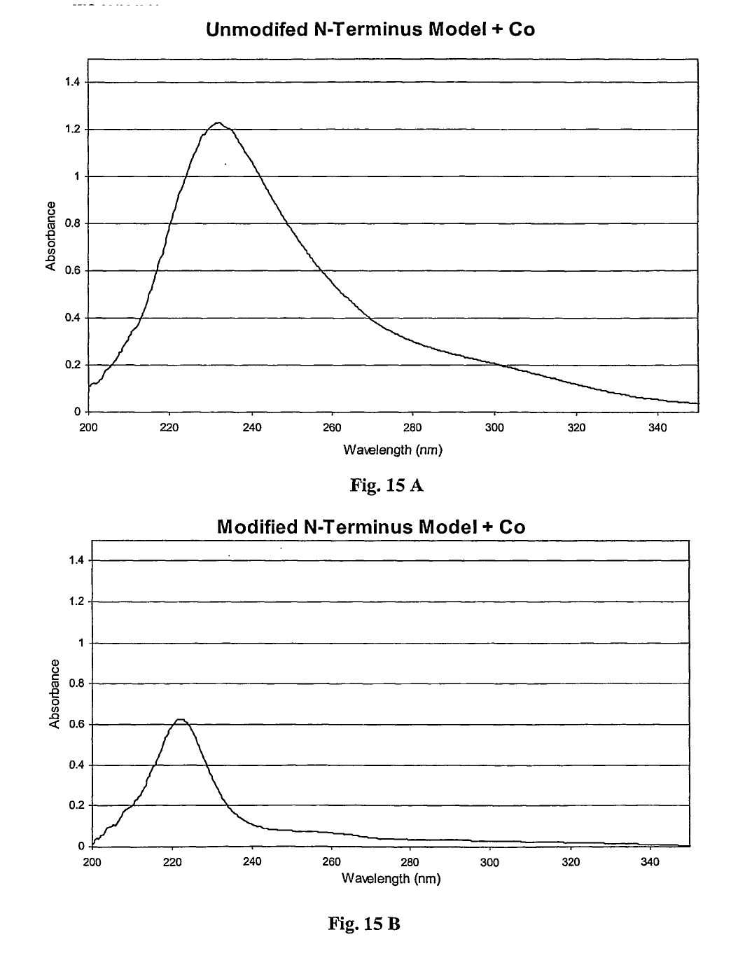

Synthetic peptide models of unmodified and modified (i.e., missing the terminal 4 amino acids) albumin N-terminus were incubated with cobalt ion and their absorption spectra were measured. Results indicate that cobalt binding significantly increases the extinction coefficient of N-terminal models as well as shifting the absorption peak from ˜220 nm to approximately ˜235 nm (see FIGS. 15A and B).

### Example 11

Quantification of Degree of Modification of Albumin N-Terminus Based on Co Binding

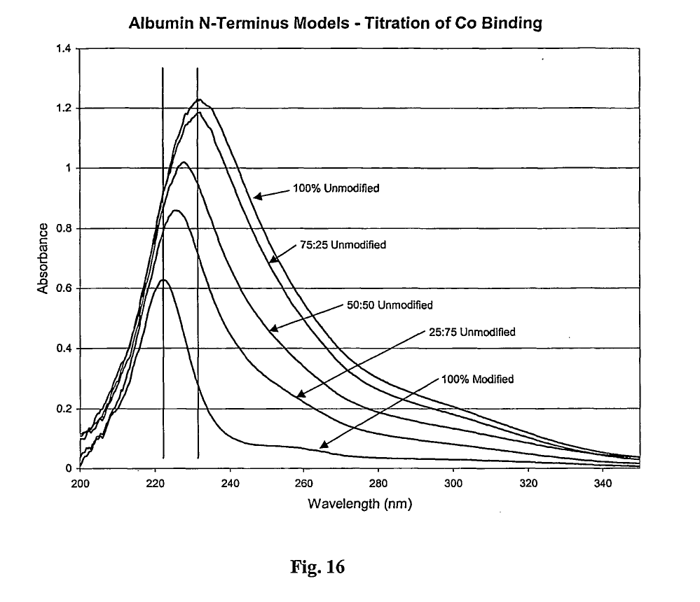

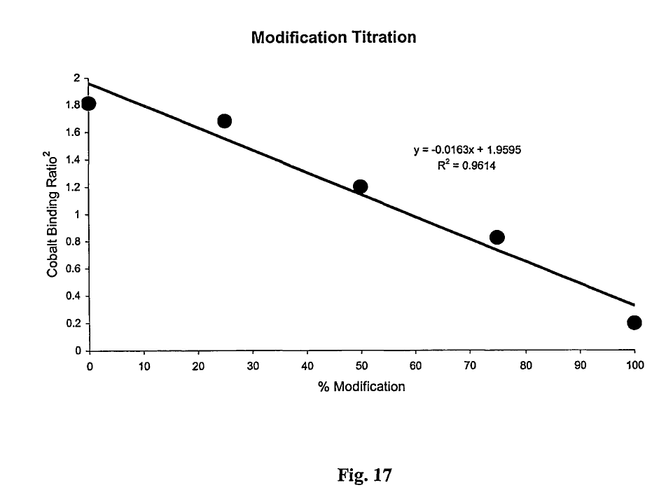

To assess the ability to quantify the cobalt ion binding spectroscopically, five mixtures of modified and unmodified N-terminus albumin models with different ratios were measured. The spectra are shown in FIG. 16. For each curve the squared ratios of the absorption at 232 nm to 221 nm were calculated, and the result correlated to the percent modification. The results are shown in FIG. 17. The results indicate that absorbance can be used to quantify amount of N-terminally modified albumin.

### Example 12

Optical Measurement of Change in Albumin N-Terminus Without Addition of a Metal Reagent

To assess the viability of spectroscopy on human albumin, normal human albumin was modified via a slow chemical reaction with an enzyme which systematically digests peptides sequentially from the N-terminus. Spectra were obtained at multiple time points, and it was observed that the wavelength where there was most change was in the region of 235 nm. Results indicate that differences of the N-terminus can be seen spectroscopically. This experiment was conducted without cobalt, providing evidence that changes in the N-terminus of albumin can be observed spectroscopically without the addition of cobalt or other reagents.

The absorbance at 235 nm was plotted against time (see FIG. 18). In this experiment, time is related to % modification, although not linearly. We observe classical enzymatic reaction kinetics with a plateau at approximately 40 minutes. The observed spectral changes without the use of reagents indicate the utility of a reagent-free test for real-time ischemia measurement. An optical probe can be placed intravenously to observe the spectrum in the region of 235 nm to monitor the level of ischemia continuously.

[image]

[image]

[image]

[image]

[image]

[image]

[image]

[image]

[image]

1. A method for detecting a medical condition comprising:
providing a patient fluid sample divided into first and second portions, and combining a substance for providing free metal ions with the first portion of the sample;
irradiating both the first and second portions of the sample with light;
determining absorbance values for the first and second portions;
obtaining a differential absorbance value from the first and second portions;
analyzing the differential absorbance value for determining one or more characteristics that are indicative of whether the medical condition is present;
wherein said analyzing step uses principal component analysis for reducing a dimension of the differential absorbance value.

2. The method of claim 1, wherein the medical condition is ischemia.

3. The method of claim 1, wherein the metal ion is cobalt ion.

4. A method of diagnosing an ischemic event comprising:
a) providing a first and second patient sample comprising albumin;
b) adding to the first patient sample a metal ion, whereby the metal ion binds to the albumin;
c) conducting optical analyses of the first and second patient samples to generate signals or spectra, respectively;
d) measuring the amount of metal bound to the albumin by comparing the signals or spectra of step (c) to generate a differential signal or spectra; and
e) comparing the differential signal or spectra to a standard curve or mathematical model that correlates the differential signal or spectra to amount of metal bound to albumin, whereby an ischemic event may be diagnosed if the measured amount of metal bound to albumin is below a defined value.

5. The method of claim 4, wherein the patient samples are serum.

6. The method of claim 4, wherein the first and second patient samples are provided by dividing an original patient sample.

7. The method of claim 43, wherein the metal ion is cobalt ion.

8. The method of claim 4, wherein the metal ion binds to the N-terminus of the albumin.

9. The method of claim 4, wherein the optical analyses are absorbance spectroscopy and the analyses is conducted in the range of 300-450 nm.

10. The method of claim 4, wherein the optical analyses comprise fluorescence spectroscopy, said method further comprising:
adding to the first patient sample a fluorescent dye in step (b), wherein the dye binds to said metal ion and wherein the fluorescence signal changes as a function of whether the metal ion is unbound or bound to the albumin.

11. The method of claim 4, further comprising:
analyzing the differential signal or spectra of step (d) using principal component analysis.

12. A method for diagnosing an ischemic event, comprising:
a) adding a metal ion and a fluorescent dye to a patient sample comprising albumin, whereby the dye binds to the metal ion which may bind to the albumin;
b) measuring the metal bound to the albumin by measuring a fluorescent signal of the sample, wherein the fluorescent signal changes as a function of whether the metal ion is unbound or bound to the albumin; and
c) comparing the fluorescent signal to a standard curve or mathematical model that correlates the fluorescent signal to an amount of metal ion bound to albumin, whereby the measurement of metal ion bound to albumin below a defined value may be diagnostic for an ischemic event.

13. The method of claim 12 wherein the metal ion and fluorescent dye are added as a conjugate.

14. The method of claim 12, wherein the fluorescent signal is quenched or shifts to a different wavelength when the dye is bound to a metal ion that is bound to the albumin.

15. The method of claim 12, wherein the metal ion is a cobalt ion.

16. The method of claim 12, wherein the fluorescent dye is selected from the group consisting of Cumarin, Rhodamine and Newport green.

17. A method of rapidly diagnosing an ischemic event comprising:
a) providing a first and second patient sample comprising albumin;
b) adding to the first patient sample a metal ion, whereby the metal ion binds to the albumin in a reaction that reaches equilibrium at a predetermined time;
c) conducting, during a defined time interval prior to achievement of equilibrium, optical analyses of the first and second patient samples to generate first and second signals or spectra, respectively, for each sample at selected time points during the defined time interval;
d) measuring the rate of change of amount of metal bound to the albumin over the defined time interval by comparing the first and second signals or spectra for each time point to generate differential signals or spectra for each time point in the time interval;
e) calculating a rate of change in the differential signals or spectra over the time interval;
f) and comparing the rate of change of signal or spectra to a standard curve or mathematical model that correlates rate of change with projected metal bound to albumin at equilibrium, whereby an ischemic event may be diagnosed if the projected amount of metal bound to albumin is below a defined value.

18. A method of rapidly diagnosing an ischemic event, comprising:
a) adding a metal ion and a fluorescent dye to a patient sample comprising albumin, whereby the dye binds to the metal ion which binds to the albumin in a reaction that reaches equilibrium at a predetermined time, wherein the fluorescent dye's signal changes as a function of whether the metal ion is unbound or bound to the albumin;
b) measuring the rate of change of metal bound to the albumin by measuring the fluorescent signal of the sample at selected time points over a time interval that is prior to achievement of equilibrium;
c) calculating the rate of change of the fluorescent signal over the time interval; and
d) comparing the rate of change of the fluorescent signal to a standard curve or mathematical model that correlates the rate of change in fluorescent signal to a projected amount of metal ion bound to albumin at equilibrium, whereby ischemia may be diagnosed if the measured rate of change of metal ion bound to albumin is below a defined value.

19. A method for diagnosing an ischemic event comprising:
(a) providing a patient sample comprising albumin, a portion of which may be N-terminally modified;
(b) measuring the N-terminally modified albumin by measuring absorbance of the sample, and comparing the absorbance to a standard curve or mathematical model that correlates the absorbance to a ratio of modified to unmodified albumin, wherein an ischemic event may be diagnosed if the ratio is below a defined value.

20. The method of claim 19, wherein the patient sample comprises whole blood, serum or plasma provided in a sample container and the absorbance is measured with a spectral probe placed in the sample.

21. The method of claim 19, wherein the patient sample comprises whole blood in the patient's blood vessel and the absorbance is measured with a spectral probe placed in the blood vessel.

22. An instrument for detecting a medical condition, comprising:
a spectral probe having a tip for insertion into a patient fluid sample and receiving spectral light from the patient fluid sample;
a spectrophotometer coupled to said spectral probe for quantifying each frequency of spectral light received by said spectral probe and outputting a signal representative of the quantity of each frequency of spectral light;
a computing unit, comprising:
an input coupled to receive the signal from said spectrophotometer;
a memory for storing a model representing spectral light data obtained from a first set of patients known to have the medical condition and a second set of individuals known to not have the medical condition, whereby the model includes a value identified with a high probability of the presence of the medical condition;
a processor programmed to execute instructions for:
comparing the quantity of each frequency of spectral light from the patient with corresponding data in the stored model; and
determining whether the quantity of each frequency of spectral light is indicative of the presence of the medical condition in the patient; and
an output to provide the determination to a user.

23. A method for providing an instrument for diagnosing a medical condition in a patient, comprising:
obtaining a control fluid sample from a first plurality of control individuals known to have the medical condition;
obtaining a control fluid sample from a second plurality of control individuals known to not have the medical condition;
dividing each control fluid sample into first and second portions;
combining a substance for providing free metal ions with the first portion of each control fluid sample;
irradiating both the first and second portions of each control fluid sample with light;
determining absorbance values for the first and second portions of each control fluid sample;
obtaining a differential absorbance value from the first and second portions of each control fluid sample;
generating a principal component analysis model of the obtained differential absorbance values, the principal component analysis model including a value indicative of the presence of the medical condition;
storing the generated principal component analysis model in a computer readable format;
providing computer executable instructions for:
providing a differential absorbance value, determined from first and second portions of a patient fluid sample obtained from a patient, said first portion having been combined with free metal ions, and comparing said differential value with the stored principal component analysis model;
in response to the comparing step, determining whether the differential absorbance value of the patient fluid sample is indicative of the presence of the medical condition.
